(19) United States
(12) Patent Application Publication
(10) Pub. No.: US 2008/0125845 A1
(43) Pub. Date: May 29, 2008

(54) PROXIMAL FIXING

(75) Inventors: Heike Fischer, Meerbusch (DE); Steffen Rauschenberger, Reutlingen (DE)

Correspondence Address: KLARQUIST SPARKMAN, LLP, 121 SW SALMON STREET, SUITE 1600, PORTLAND, OR, 97204, US

(73) Assignee: JOTEC GmbH

(21) Appl. No.: 11/946,590

(22) Filed: Nov. 28, 2007

(30) Foreign Application Priority Data
Nov. 29, 2006 (DE) 10 2006 058 186.5

(51) Int. Cl. A61F 2/82

(52) U.S. Cl. 623/1.2

(57) ABSTRACT

The present invention relates to a system for inserting a self-expanding stent into a vessel of the body, with a proximal area and a distal area, and with a self-expanding stent which has a hollow cylindrical body with a proximal end and a distal end. At least the proximal end has meshes. The system further comprises a detachable fixing system for the proximal end of the stent, for inserting the stent into the vessel, wherein the fixing system consists of a wire-like element which has a first end and a second end, and a portion lying between these, and by which the meshes can be converted from an expanded state to a compressed state for inserting the stent into the vessel.

### CROSS REFERENCES TO RELATED APPLICATION

This application claims priority from German patent application DE 10 2006 058 186.5, filed Nov. 29, 2006.

### BACKGROUND OF THE INVENTION

The present invention relates to a system for inserting a self-expanding stent into a vessel of the body, with a proximal area and a distal area, and with a self-expanding stent which has a hollow cylindrical body with a proximal end and a distal end, at least the proximal end having meshes, and the system comprising a detachable fixing system for the meshes of the proximal end of the stent, for inserting the stent into the vessel.

Vascular stents, also called endovascular stents, are inserted into a diseased vessel in order to treat aneurysms, dissections, ruptures or stenoses, and are described in detail in the prior art.

They are used for bridging the diseased area of the vessel. In addition to having a main framework, in most cases of wire, a stent graft also has a jacket made of biocompatible material.

Stents or stent grafts known from the prior art are inserted, for example, into vessels whose walls are weakened by disease or by injury and which require support for that reason.

Many stents take the form of self-expanding stent systems which are inserted in the compressed state into the vessel and whose expansion is permitted by removal of compressing means. It is therefore necessary for these self-expanding stents to comprise an elastic material that can expand outward, i.e. radially, as soon as a compression force exerted on the material, for example by a sleeve, is removed. A material with superelastic properties is preferably used here. The stent framework is made from this material and preferably has a tubular structure which in most cases has a slightly larger diameter than the vessel into which it is to be introduced.

The insertion and placement of a stent or stent graft is usually performed using an insertion system in which three tubular structures are provided, namely a guidewire catheter, an inner piston and an outer sleeve, which can be moved axially relative to one another. The stent is arranged in the compressed state inside the distal end of the outer sleeve and is inserted into the vessel in this state. After the stent has been placed at the desired location, the piston is in most cases held stationary, whereas the sleeve of the insertion system is pulled back, as a result of which the stent is released. Because it makes contact with the piston, the stent cannot move in the direction of the withdrawing sleeve when the latter is removed. By virtue of its self-expanding property, the stent unfolds and rests at least partially on the vessel walls.

In the prior art, the end of the stent placed closer to the heart is generally called the proximal end, whereas the end of the stent placed further away from the heart is called the distal end. In contrast, the designation of the ends of the insertion system as distal and proximal is such that the end nearer to the operator is called the proximal end, and the end further away from the operator is called the distal end.

The proximal end of the stent or stent graft is typically designed such that the stent is fixed mainly with this end on the vessel wall. This is intended to prevent displacement of the stent after it has been introduced into the vessel. For this purpose, the ends of the stent comprise spring elements which form circumferentially meandering loops or an undulating ring, which elements in the present case are generally designated as meshes and expand radially after their release and rest on the vessel wall as fixing elements. In many cases, small hooks are also provided at the proximal end of the jacket and, after release, penetrate into the vessel wall and additionally fix the implant. In the expanded state, the proximal end with these fixing elements in most cases has a larger diameter than the vessel into which the stent is intended to be introduced, specifically to ensure that the fixing elements at the proximal end of the stent bear firmly on the vessel walls, after release of the stent, and are able to anchor themselves there. Therefore, these fixing elements at the proximal end of the stent have to be compressed, to permit insertion of the stent into the vessel of the body, and have to be secured releasably in the insertion system.

In many stents known in the prior art, in addition to the stent the proximal end of the stent is also compressed by a sleeve tube, which holds the stent in a compressed state for insertion into the vessel. When the stent or stent graft is released, it expands, starting at the proximal end and the fixing elements and going toward the distal end.

On the other hand, the prior art also discloses insertion systems which permit separate release of the proximal end of the stent from the rest of the stent. In this way, the fixing elements can be released only when the rest of the stent or stent graft has already been partially or completely expanded.

Upon release of the stent or stent graft, a slight movement of the partially expanded stent graft in the proximal or distal direction may be necessary in order to ensure the best possible positioning. This secondary correction of the stent position is, however, associated with a very considerable risk of injury of the vessel walls, if the loops/meshes of the proximal end of the stent are already opened and hooked in the vessel wall.

### RELATED PRIOR ART

To solve this problem, the prior art discloses, for example, the insertion system known from EP 1 369 098 A1, which has a cap inside which the proximal spring ends of the stent are guided and secured and the proximal spring ends of the stent graft are released by a mechanism in the cap or by removal of the cap only after the positioning.

WO 2005/023149 discloses an insertion system for stents or stent grafts in which the springs of the stent are brought together by a capturing device for the loops of the springs. This capturing device has a suitable number of fixed elongate projections into which the springs are threaded. The projections are fixed securely on an outer tube or catheter which is guided through the stent lumen. By pulling the tube back, i.e. by pulling the tube in the direction of the operator, and the projections fixed securely on said tube, the threaded springs of the proximal stent end are released.

A disadvantage of the systems known in the prior art is that with these systems there is a danger that, if the springs get caught on the system fixing them or if the springs do not fully release upon withdrawal of the capturing device, the proximal end of the stent may not be released. The system known from WO 2005/023149 also has the disadvantage that twisting of the outer catheter also entails a risk of twisting of the stent.

A further disadvantage of the devices known in the prior art is that the releasing and fixing systems of these devices are in several parts and are mostly of very complicated construction, which makes the system as a whole expensive and prone to failure. Moreover, the components have the great disadvantage that they greatly stiffen the proximal area of the insertion system, which makes insertion and removal of the insertion system through the narrow access vessels and in the aortic arch considerably difficult or impossible. A further disadvantage of the already known fixing systems is that they take up space, in addition to the implant material, and thus do not satisfy the aim of making the diameter of the insertion equipment as small as possible.

### SUMMARY OF THE INVENTION

An object of the present invention is therefore to make available a system for inserting a self-expanding stent into a vessel of the body, which system is able to overcome the known disadvantages of the prior art.

According to the invention, this object is achieved by developing the insertion system mentioned at the outset, wherein the fixing system consists of a single wire-like element by which the meshes can be converted from an expanded state to a compressed state for inserting the stent or stent graft into the vessel.

The object of the invention is achieved in full in this way. With the insertion system according to the invention and in particular with the fixing system contained therein for the proximal end of the stent, it is now possible to release the meshes by pulling the wire, without the risk of these becoming caught in the release system. Moreover, the wire-like element affords an extremely simple solution in which the proximal end of the stent can be released without other parts or devices having to be provided in the system for fixing the meshes. In this way, the system as a whole is very easy to handle, is less prone to failure, is highly flexible and saves space, i.e. permits small diameters of the insertion system.

In the system according to the invention for inserting the stent into a vessel of the body, the proximal end of the stent or as the case may be the meshes present at the proximal end are practically threaded on with the wire-like element and thereby gathered together or compressed in the manner of meshes. The diameter of the proximal end of the stent is thus considerably reduced, compared to the expanded state of the end of the stent, thereby permitting easy insertion into the vessel. To release and secure the stent in the vessel, the wire-like element is simply removed by moving it in the direction of the operator, that is in the direction of the proximal end of the stent, for example by simply pulling the wire-like element out of the meshes, as a result of which the meshes are released by virtue of the self-expanding property of the stent.

It will be appreciated that the wire-like element must have properties which, on the one hand, allow the stent to be held in the compressed state and which, on the other hand, allow the wire-like element to be easily pulled back out of the meshes in order to release them. Further, the wire-like element can be completely removed from the stent and the delivery system after having released the meshes.

In the present context, “meshes” refers to any structure which, at the proximal end of the stent, is composed of openings and of filament-like or wire-like elements surrounding these openings. The meshes can be of different shapes, for example annular or circular, or in the form of arches/loops. The fixing system at the proximal end of the stent can be formed, for example, by a circumferentially meandering wire element, or by separate rings that are sewn at a later stage onto the proximal end of a stent or stent graft.

In the present context, “stent” is intended to designate any device which, with a latticework serving as hollow cylindrical body, is introduced into vessels in order to support their walls.

Therefore, in the present context, the term stent also includes stent grafts which, in addition to having an expandable main frame, also have a jacket which at least partially covers the latter. Moreover, the stent can assume any desired shape, for example a tubular shape, or the shape of a bifurcation, or the shape of a tube with lateral branches.

The functional wire-like element can, for example, have a diameter of ca. 0.1 mm to ca. 1 mm, preferably from ca. 0.3 mm to ca. 0.35 mm, and a length of between ca. 1 cm and ca. 300 cm.

The wire-like element, at least in the functional area or over the entire length of the wire, has a material with shape-memory properties, in particular nitinol with shape-memory properties under the conditions prevailing in clinical application.

The material called nitinol is of course already known per se in applications for stents. Nitinol is often employed here with superelastic properties, as a result of which stents can be produced that can be converted from an expanded state of rest to a loaded compressed state. After the load or compression is removed, the stent, by virtue of its superelastic properties, expands back to the original state of rest, which means that, when it is released in a vessel in the body for example, it bears on the vessel walls as a result of the expansion.

In one embodiment of the system according to the invention, provision is made for the fixing system at the proximal end of the stent to be a nitinol wire with a shape-memory property which is threaded through the meshes and thus fitted releasably in the stent, and which, in order to release the stent, can be withdrawn from the meshes in order to release the stent in a vessel. The shape-memory properties of the nitinol wire ensure that the wire has sufficient strength for holding the meshes in the compressed state, in order to permit insertion of the stent, while on the other hand ensuring that it is still easy to remove the nitinol wire from the meshes, such that they are no longer held together and are thus released and can bear on the wall of the vessel.

In another embodiment, the wire-like element for fixing the meshes is guided alternately from the inside out through the meshes, and optionally through the first mesh at the end again.

This measure has the advantage that the meshes can be easily threaded onto the wire-like element and gathered together. The diameter of the proximal end of the stent is thereby reduced as a whole compared to the unloaded state.

It will be appreciated that the shape of the proximal end of the stent or stent graft depends on the different stents and on the purpose for which they are used. Because of the way they are produced, the ends of most stents have meshes or loops, the number of these varying between two and several, for example 5, 6, 7 or 8, likewise depending on the stent type and stent size and on its intended use. In the fixing system according to the invention, one advantage is that it is irrelevant how many loops the proximal end of the stent comprises, since, in order to compress the diameter of the proximal end of the stent, the meshes or loops provided at this end can be easily threaded onto the wire-like element, without having to take into account the exact number of meshes or loops. The wire-like element can therefore be used in principle in all stents which, at their proximal end, have a mesh structure through which the wire-like element can be guided. It will be appreciated that the system may be employed with human beings being in need of a vessel support, as specified in the outset, and also with other manuals.

In another embodiment, the proximal end of the stent can be fixed with the first end of the wire-like element, and the intermediate portion and the second end of the wire-like element are guided to the proximal area of the insertion system.

This embodiment affords the advantage that the second end is guided to the person operating the insertion system, as a result of which said person is able to influence the fixing system at the distal end of the insertion system.

According to another embodiment, the first end of the wire-like element can be removed from the proximal end of the stent by exerting a pull on the second end of the wire-like element.

This embodiment has the advantage that, with the second end of the wire-like element present in the proximal area of the insertion system, the fixing system can be released from the proximal end of the stent by a simple maneuver. By exerting a pulling force on the second end of the wire-like element, the latter is moved counter to the direction of insertion of the stent, as a result of which the first end of the wire-like element is also pulled out of the meshes or loops of the proximal end of the stent. In this way, the meshes or loops are in turn released and are able to expand. Therefore, only a single maneuver is needed here in order to release the proximal end of the stent.

It will be appreciated that the second end can be guided for example through the hollow body of the stent. If appropriate, it can also be guided outside of the stent.

In one embodiment of the system according to the invention, the meshes at the proximal end of the stent are loops which point alternately in the proximal and distal directions and which comprise a vertex and limbs.

In this embodiment, therefore, at least the proximal end of the stent is composed of springs shaped as loops. In order to fix the loops, the wire-like element is guided from the inside outward through the loops and tightened, as a result of which they can be fixed and compressed.

In another embodiment, the system for inserting a self-expanding stent is provided with tubular portions through which the wire-like element is guided along the guidewire catheter. These tubular portions can for example be located on a guidewire catheter which is normally provided in an insertion system for stents. These tube portions are preferably made of plastic and can be designed in the form of a double-lumen tube, one lumen serving as a guide for the wire-like element, and the second lumen being used for stopping on the guidewire catheter. A tube portion is in this case positioned on the guidewire catheter such that the proximal end terminates for example ca. 0 to 5 mm below the fixed loops, so as to prevent the loops being moved along the guidewire catheter when the wire is pulled.

In another embodiment, the system further comprises a withdrawal sleeve which holds at least parts of the stent in a compressed state for insertion into a vessel.

As has been mentioned further above, the proximal end of the stent can be released in a targeted manner via the fixing system. The remaining portions of the stent can be compressed, independently of the proximal end of the stent, for inserting the stent into a vessel in the body, by means of a force exerted via a sleeve pulled over the stent, and they can be released by pulling the sleeve back. In this way, the stent can be fully expanded and anchor itself in the vessel.

Moreover, in another embodiment, a pusher element is provided by means of which the distal end of the stent and the part of the stent lying between the distal end and proximal end can be released in conjunction with a withdrawal of the withdrawal sleeve.

With the pusher element, it is possible to counteract the force that is applied to the stent by removal of the sleeve. This means that the stent does not move together with the withdrawal sleeve in the proximal direction relative to the insertion system, but can instead be held at the position where it was originally intended to be released.

The system can also have features and properties customary in stent release systems. Reference may be made for example to systems such as are described in DE 103 46 200, the disclosure of which is herewith incorporated by reference.

The invention further relates generally to the use of a wire-like element with which fixing elements pointing in the proximal and distal directions can be fixed releasably at the proximal end or distal end of a stent.

In particular, it is preferable here if the wire-like element has shape-memory properties under the conditions in which it is used, and still more preferable if the wire-like element is a nitinol wire with shape-memory properties under the conditions of use.

The inventor has recognized that, in order to fix proximal ends of stents, it is generally advantageous to use a wire with which the stent end comprising meshes or openings can as it were be threaded on and thereby compressed in diameter. In this state, the stent can then be easily inserted into a vessel in the body. In order to release the proximal end of the stent, the wire is simply removed from the meshes/openings or loops and pulled out lengthwise, as a result of which these are able to expand again.

It will be appreciated that the features mentioned above and to be explained in more detail below can be used not only in the respectively cited combination, but also singly or in other combinations, without departing from the scope of the present invention.

### BRIEF DESCRIPTION OF THE DRAWINGS

Further advantages will become evident from the following detailed description of the invention and from the attached drawing, in which:

FIG. 1 shows an example of a stent (graft) in its expanded form, with the proximal end of the stent released (not true to scale);

FIG. 2 shows the proximal end of the stent, the loops of the proximal end being held together here by the fixing system of the inventive embodiment for inserting the stent into a vessel of the body (not true to scale); and

FIG. 3 shows the release of the proximal end of the stent, with the fixing system partially pulled out (not true to scale).

### DESCRIPTION OF PREFERRED EMBODIMENTS

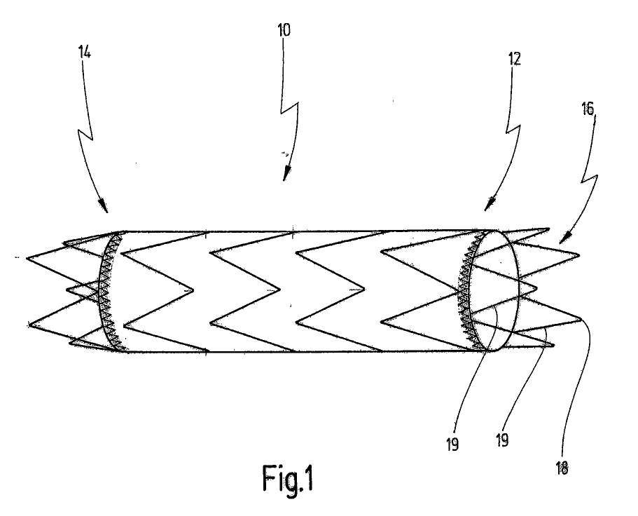

An example of a stent 10 is shown in FIG. 1, with a cylindrical hollow body and with a proximal end 12 and distal end 14. The proximal end 12 of the stent has loops, which are indicated overall by 16 in FIG. 1. These loops 16 have vertices 18 and straight portions or limbs 19.

The stent in FIG. 1 is covered in its central portion by a jacket, which holds the stent tight in the inserted state. The stent (or stent graft) shown in FIG. 1 is in its expanded state, which is also referred to herein as its rest state or as the unloaded state, since the stent assumes this shape after its production. The stent is further composed of meandering circumferential rings which form respective loops pointing alternately in the proximal and distal directions.

In general, the stent usually has a slightly larger diameter than the vessel of the body into which it is intended to be implanted. The stent can also have different diameters along its length. For example, the proximal end and distal end can have larger diameters than the stent area located between the proximal and distal ends. Moreover, the loops can additionally be spread outward in a crown shape. The mesh size, the height of the individual stent springs, and the number of loops can also vary along the length of the stent.

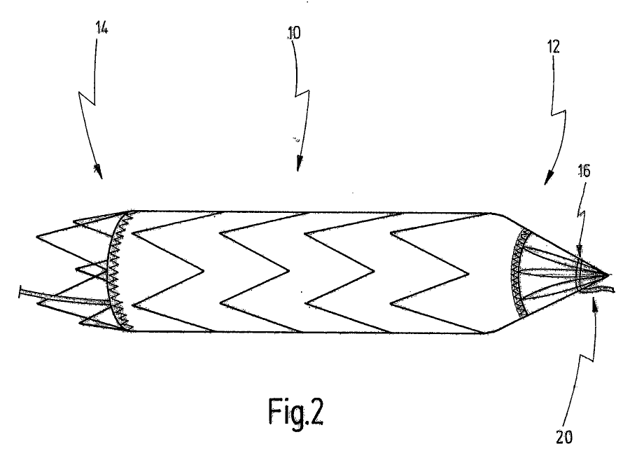

In FIG. 2, the stent shown in FIG. 1 is shown partially in another state. Here, identical features are designated by the same reference numbers. Reference number 20 designates a wire-like element which is threaded into the loops 16 or onto which the meshes or loops are as it were threaded. In this way, the loops 16 can be gathered together, as a result of which the diameter at the proximal end 12 of the stent 10 decreases.

The rest of the stent can now be compressed too, for example by engagement of a withdrawal sleeve over it, as a result of which the diameter of the remaining portions of the stent also decreases. In this compressed and loaded state, the stent can be introduced into a vessel of the body. For this purpose, the system for inserting the stent can comprise further component parts, for example a pusher, a guidewire catheter, an inner tube onto which the stent is loaded, etc. These elements are not shown in the figures, since they are conventional elements of insertion systems and will be clear and evident to a person skilled in the art on reading through the present application.

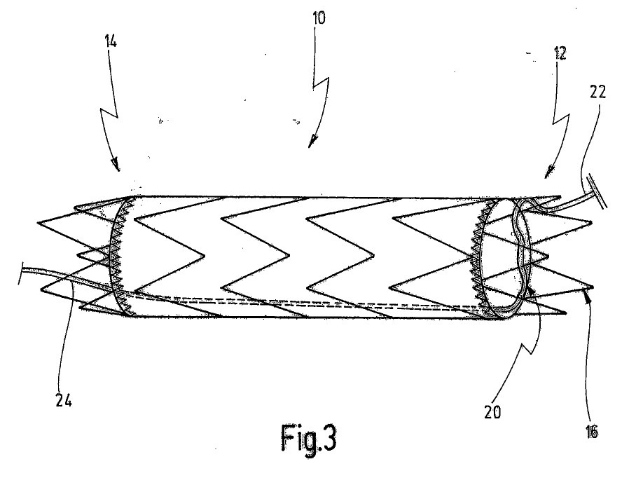

FIG. 3 again shows the stent from FIGS. 1 and 2, this time in a third state. Here too, identical features are again provided with the same reference numbers as in FIGS. 1 and 2. As will be seen from FIG. 3, the first end 22 of the wire-like element 20 holds the loops together, while the second end 24 of the wire-like element 20, or the portion of the wire-like element 20 located between the first end 22 and second end 24, is guided through the lumen of the stent, as is indicated by way of example by the broken line in FIG. 3. The wire-like element 20 in FIG. 3 has been partly removed from the loops 16, for example by pulling on the second end 24 of the wire-like element 20, as a result of which the proximal end 12 of the stent 10 is released again.

To completely release the stent, the withdrawal sleeve is preferably partially pulled back, the wire-like element 20 is removed completely from the proximal end 12 of the stent, and the withdrawal sleeve, which is not shown in FIGS. 1 to 3, is then pulled back completely.

The stent shown in the figures has been chosen only as an example. It will be appreciated that any other form of a stent or stent graft with different proximal ends can be used, or at least any stent/stent graft having mesh-like or ring-like elements at its proximal end. These do not have to be an integral part of the stent. For example, they can also be subsequently sewn onto a stent or stent graft and thus permit the proximal fixing. Moreover, the wire-like element in a stent graft could also be threaded through the jacket and draw the stent together at the front if there are no free stent springs/meshes present.

Therefore, what is claimed is:

1. A system for inserting a self-expanding stent into a vessel of the body, the system having a proximal area and a distal area, and comprising a self-expanding stent which has a hollow cylindrical body with a proximal end and at least one distal end, at least the proximal end having meshes, and the system comprising a detachable fixing system for the proximal end of the stent, for inserting the stent into the vessel, wherein the fixing system consists of a wire-like element which has a first end and a second end, and a portion lying between these, and by which the proximal end of the stent can be converted from an expanded state to a compressed state for inserting the stent into the vessel, and by which the proximal end of the stent can be converted from a compressed state to an expanded state by moving the wire-like element in the direction of the proximal area of the system.

2. The system as claimed in claim 1, wherein the wire-like element has a material with a shape-memory property under the conditions of use.

3. The system as claimed in claim 1, wherein the material is shape-memory nitinol.

4. The system as claimed in claim 1, wherein the wire-like element for fixing the proximal end of the stent is guided alternately in and out through the meshes.

5. The system as claimed in claim 1, wherein the meshes can be converted to the expanded state by removal of the wire-like element.

6. The system as claimed in claim 1, wherein the proximal end of the stent can be fixed with the first end of the wire-like element, and the intermediate portion and the second end of the wire-like element are guided to the proximal area of the system.

7. The system as claimed in claim 3, wherein the first end of the wire-like element can be removed from the proximal end of the stent by exerting a pull on the second end of the wire-like element.

8. The system as claimed in claim 1, wherein the meshes at the proximal end of the stent are loops which point alternately in the proximal and distal directions and which comprise a vertex and limbs.

9. The system as claimed in claim 1, wherein a withdrawal sleeve is also provided which holds at least parts of the stent in a compressed state for insertion into a vessel.

10. The system as claimed in claim 1, wherein a pusher element is also provided by means of which the distal end of the stent and the part of the stent lying between the distal end and proximal end can be released in conjunction with a withdrawal of the withdrawal sleeve.

11. Method for fixing the proximal end of a self-expanding stent in a compassed state in order to introduce the stent in a body vessel, the method comprising the step of employing a wire-like element to keep the proximal end of the stent in a compressed state.

12. Method for delivering a self-expanding stent into a body vessel, the method comprising the steps of
fixing the proximal end of a stent into a compressed state by means of a wire-like element to introduce said stent into the vessel;
positioning the stent in the vessel at the place the vessel is to be supported; and
removing the wire-like element from the proximal end of the stent by moving it in the direction of operation in order to convert the proximal end of the stent from its compressed state into an expanded state.
